(19) United States
(12) Patent Application Publication
(10) Pub. No.: US 2012/0035275 A1
(43) Pub. Date: Feb. 9, 2012

(54) Emulsion, Method For Producing The Same, And Cosmetic Raw Material Formed From The Same

(76) Inventors: Kazuhiko Kojima, Ichihara-shi (JP); Tsutomu Naganawa, Ichihara-shi (JP); Asao Harashima, Ichihara-shi (JP); Tadashi Okawa, Ichihara-shi (JP)

(21) Appl. No.: 13/140,305

(22) PCT Filed: Dec. 21, 2009
(86) PCT No.: PCT/JP09/71831

§ 371 Date: Jun. 16, 2011

(30) Foreign Application Priority Data
Dec. 22, 2008 (JP) JP 2008-326578

(51) Int. Cl. A61K 8/891; C10M 139/04; C09K 3/00; C09D 7/12

(52) U.S. Cl. 514/772; 106/2; 106/38.22; 508/207; 252/8.57; 106/287.14; 516/124

(57) ABSTRACT

The present invention has objectives to provide an emulsion suitable as a cosmetic raw material and to easily obtain the aforementioned emulsion of organopolysiloxane exhibiting superior storage stability. The present invention provides an emulsion comprising (A) an organopolysiloxane, (B) a methyl polyglycerol-modified silicone-based surfactant having a specified structure, and (C) an aqueous medium, and provides a method for producing an emulsion characterized by obtaining an emulsion by emulsifying a mixture of (A) a organopolysiloxane and (B) a silicone-based surfactant, which is obtained by synthesizing the silicone-based surfactant of component (B) in the component (A).

### TECHNICAL FIELD

The present invention relates to an emulsion exhibiting superior handling properties, increased environmental compatibility and superior storage stability, and relates to a novel emulsification method for obtaining the aforementioned emulsion. In addition, the present invention relates to a cosmetic raw material comprising or consisting of the aforementioned emulsion.

Priority is claimed on Japanese Patent Application No. 2008-326578, filed on Dec. 22, 2008, the content of which is incorporated herein by reference.

### BACKGROUND ART

In general, various types of emulsions in the form of an oil-in-water emulsion or a water-in-oil emulsion have been utilized in wide fields of fiber treatment agents, paints, releasing agents, cosmetics and the like. The aforementioned emulsions are usually produced by emulsifying with various surfactants such as nonionic, anionic, cationic, and amphoteric ionic surfactants, having higher hydrocarbon groups, in many cases.

However, good compatibility between the hydrophobic groups of the aforementioned surfactants and organopolysiloxanes is not necessarily exhibited. For this reason, in many cases of emulsifying organopolysiloxanes, with the aforementioned surfactants, there is a problem in which poor storage stability of emulsions may be obtained if an emulsifier with a high shearing force is not used. Therefore, in order to solve the aforementioned problem, a method for emulsifying with a silicone-based surfactant such as a polyglycerol-modified polysiloxane, a polyether-modified polysiloxane or the like having a siloxane as a hydrophobic group has been proposed (see Patent Document 1 to Patent Document 4).

However, even in the case of using the polyether-modified polysiloxane, stability of the obtained emulsion was not sufficient yet. Therefore, improvements in stability of the emulsion by means of using together with another surfactant, or using a special emulsifying method, have also been proposed (see Patent Document 5 to Patent Document 8). However, there is a disadvantage in that usage is limited. Therefore, a process for easily producing an emulsion, and in particular, an emulsion of a polyorganosiloxane, exhibiting superior storage stability has been desirable.

In addition, a polyglycerol-modified polysiloxane generally has increased viscosity and is difficult to be handled.

In addition, it is reported that a polyether-modified (poly)siloxane is easily oxidized in air, and carbonyl-functional allergenic compounds such as formates and aldehydes such as formaldehyde are produced during storage over time (see Non-Patent Documents 1 to 6).
[Patent Document 1] Japanese Unexamined Patent Application, First Publication No. S61-212321
[Patent Document 2] Japanese Unexamined Patent Application, First Publication No. H6-145524
[Patent Document 3] Japanese Unexamined Patent Application, First Publication No. 2000-086437
[Patent Document 4] Japanese Unexamined Patent Application, First Publication No. S57-149290
[Patent Document 5] Japanese Unexamined Patent Application, First Publication No. H6-234918
[Patent Document 6] Japanese Unexamined Patent Application, First Publication No. H7-133354
[Patent Document 7] Japanese Unexamined Patent Application, First Publication No. H11-148010
[Patent Document 8] Japanese Unexamined Patent Application, First Publication No. H11-148011
[Non-Patent Document 1] Acta Dermato-Venereologica, 79, 5-26 (1999)
[Non-Patent Document 2] J Pharm Sci, 87, 276 (1998)
[Non-Patent Document 3] Contact Dermatitis, 44, 207 (2001)
[Non-Patent Document 4] Contact Dermatitis, 39, 14 (1998)
[Non-Patent Document 5] J Pharm Sci, 88, 4 (1999)
[Non-Patent Document 6] Contact Dermatitis, 44, 207-212, 2001

### DISCLOSURE OF THE INVENTION

### Problems to be Solved by the Invention

The present invention has a first objective to provide an emulsion of an organopolysiloxane with superior storage stability, reduced viscosity exhibiting superior handling properties, and increased environmental compatibility since it is difficult to produce allergenic compounds such as formates and aldehydes such as formaldehyde over time during storage. In addition, the present invention has a second objective to provide a preparation method in which the aforementioned emulsion can be easily produced.

### Means for Solving the Problems

The aforementioned first objective can be achieved by an emulsion comprising:
(A) an organopolysiloxane;
(B) a silicone-based surfactant represented by the following general formula (1) :

R²₃SiO(R¹₂SiO)ₘ(R¹YSiO)ₙSiR²₃ (1)

wherein
each R¹ independently represents a hydrogen atom or a substituted or non-substituted monovalent hydrocarbon group;
each Y independently represents a group represented by the following general formula (2):

—CₐH₂ₐ(OC₂H₄)_{b}(OC₃H₆)_{c}—O—(B)_{d}-A (2)

wherein
A represents a terminal group represented by the following formula (3), (4) or (5):

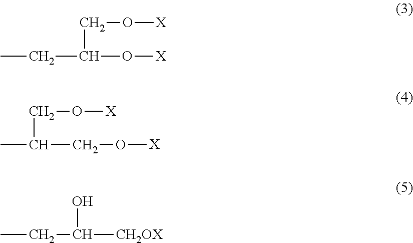

in each of the formulae, X represents a hydrogen atom or independently represents a substituted or non-substituted monovalent hydrocarbon group containing no aliphatic unsaturated bond, with not more than 20 carbon atoms; and at least one of the Xs is the aforementioned hydrocarbon group;
B represents a moiety represented by the following formula (6), (7), (8) or (9):

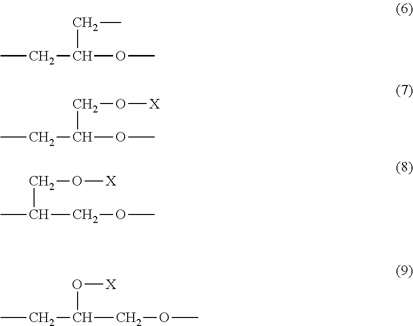

wherein
X is the same as described above;
(OC₂H₄) and (OC₃H₆) are arranged in any one of a random type, a block type, and an alternative type, or a mixed type thereof;
a ranges from 2 to 15;
b ranges from 0 to 100;
c ranges from 0 to 100; and
d ranges from 0 to 500,
m ranges from 0 to 50;
n ranges from 0 to 20; and
R² represents R¹ or X, with the proviso that when n is 0, at least one R² represents X; and
(C) an aqueous medium.

In addition, the second objective of the present invention can be achieved by a method for producing an emulsion characterized by comprising emulsifying a mixture of an organopolysiloxane (A) and a silicone-based surfactant (B), which is obtained by synthesizing the aforementioned silicone-based surfactant (B) in the aforementioned organopolysiloxane (A), wherein the aforementioned silicone-based surfactant (B) is represented by the following general formula (1):

R²₃SiO(R¹₂SiO)ₘ(R¹YSiO)ₙSiR²₃ (1)

wherein
each R¹ independently represents a hydrogen atom or a substituted or non-substituted monovalent hydrocarbon group;
each Y independently represents a group represented by the following general formula (2):

—CₐH₂ₐ(OC₂H₄)_{b}(OC₃H₆)_{c}—O—(B)_{d}-A (2)

wherein
A represents a terminal group represented by the following formula (3), (4) or (5):

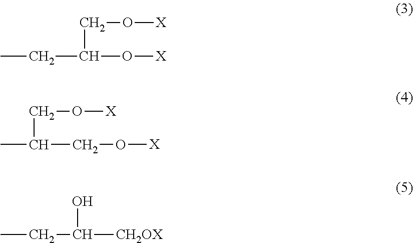

in each of the formulae, X represents a hydrogen atom or independently represents a substituted or non-substituted monovalent hydrocarbon group containing no aliphatic unsaturated bond, with not more than 20 carbon atoms; and at least one of the Xs is the aforementioned hydrocarbon group;
B represents a moiety represented by the following formula (6), (7), (8) or (9):

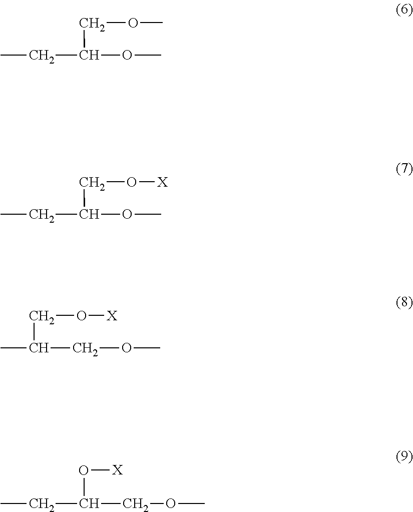

wherein
X is the same as described above;
(OC₂H₄) and (OC₃H₆) are arranged in any one of a random type, a block type, and an alternative type, or a mixed type thereof;
a ranges from 2 to 15;
b ranges from 0 to 100;
c ranges from 0 to 100; and
d ranges from 0 to 500,
m ranges from 0 to 50;
n ranges from 0 to 20; and
R² represents R¹ or X, with the proviso that when n is 0, at least one R² represents X.

In the aforementioned general formula (1), m preferably ranges from 0 to 6 and n preferably ranges from 0 to 3. In addition, at least 15% of the Xs of the aforementioned terminal groups is preferably the aforementioned hydrocarbon group.

In addition, the aforementioned silicone-based surfactant (B) is preferably synthesized by subjecting a silicon atom-bonding hydrogen atom-containing siloxane and a terminal double bond-containing compound to a hydrosilylation reaction in the presence of a catalyst for use in a hydrosilylation reaction.

The aforementioned silicon atom-bonding hydrogen atom-containing siloxane can be represented by the following general formula (1′):

R²₃SiO(R¹₂SiO)ₘ(R¹HSiO)ₙSiR²₃ (1′)

wherein each R¹ independently represents a hydrogen atom or a substituted or non-substituted monovalent hydrocarbon group;
m ranges from 0 to 50;
n ranges from 0 to 20; and
R² represents R¹ or H, with the proviso that when n is 0, at least one R² represents H. In the aforementioned general formula (1′), m preferably ranges from 0 to 6 and n preferably ranges from 0 to 3.

The aforementioned terminal double bond-containing compound can be represented by the following general formula (2′):

CH₂═CH—C_{a′}H_{2a′}(OC₂H₄)_{b}(OC₃H₆)_{c}—O—(B)_{d}-A (2′)

wherein
A represents a terminal group represented by the following formula (3), (4) or (5):

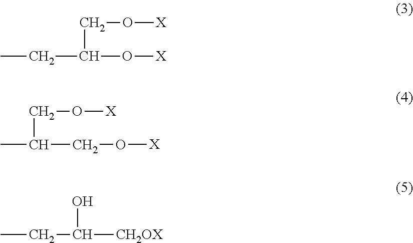

in each of the formulae, X represents a hydrogen atom or independently represents a substituted or non-substituted monovalent hydrocarbon group containing no aliphatic unsaturated bond, with not more than 20 carbon atoms; and at least one of the Xs is the aforementioned hydrocarbon group;
B represents a moiety represented by the following formula (6), (7), (8) or (9):

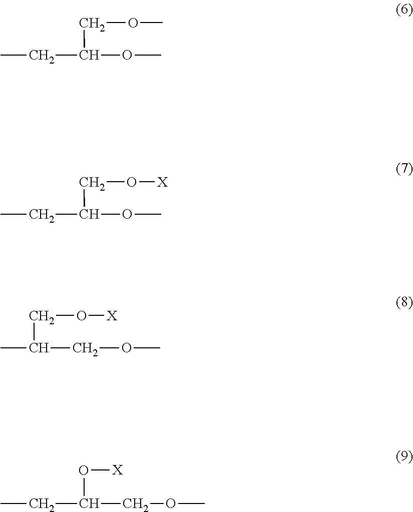

wherein
X is the same as described above;
(OC₂H₄) and (OC₃H₆) are arranged in any one of a random type, a block type, and an alternative type, or a mixed type thereof;
a′ ranges from 0 to 13;
b ranges from 0 to 100;
c ranges from 0 to 100; and
d ranges from 0 to 500.
   In addition, at least 15% of the Xs of the aforementioned terminal groups is preferably the aforementioned hydrocarbon group.

The viscosity of the aforementioned organopolysiloxane (A) preferably ranges from 50 to 3,000 mPa·s at 25° C.

In addition, the present invention relates to an emulsion obtainable by the aforementioned preparation method.

### Effects of the Invention

In accordance with the present invention, an emulsion of an organopolysiloxane exhibiting superior handling properties, superior environmental compatibility, and superior storage stability can be provided, and a preparation method in which the aforementioned emulsion can be easily obtained can also be provided.

Namely, in the aforementioned silicone-based surfactant (B) used in the present invention, terminal OH groups are partially alkylated, and for this reaction, hydrogen bonding between the aforementioned OH groups can be controlled. Therefore, the aforementioned silicone-based surfactant (B) has reduced viscosity, and the emulsion containing the same also has reduced viscosity and exhibits superior handling properties. Therefore, the emulsion obtained in accordance with the present invention is easily blended in a cosmetic or the like.

In addition, the aforementioned silicone-based surfactant (B) used in the present invention is difficult to be oxidized in air, and allergenic compounds such as formates, aldehydes such as formaldehydes have difficultly being produced over time during storage, as compared with a conventional polyether-modified (poly)siloxane. For this reason, the emulsions obtained in accordance with the present invention exhibit increased environmental compatibility even if an after-treatment such as a hydrogenation treatment, addition of antioxidants or the like is not carried out. Therefore, the emulsions obtained in accordance with the present invention can be suitably used, in particular, in a cosmetic or the like, which is used on human beings. In this case, the aforementioned cosmetics can be used for a long period of time. In addition, it is not necessary to add additives such as an antioxidant and the like in order to prevent generation of allergenic compounds. For this reason, a cosmetic having a more natural composition can be formed.

In particular, in the case of producing the aforementioned silicone-based surfactant (B) by means of a hydrosilylation reaction, the terminal group bonds to a polysiloxane not by an Si—O—C bond, but by an Si—C bond. Therefore, the aforementioned silicone-based surfactant (B) exhibits reduced hydrolysis decomposition properties, is stable over time, and can maintain the aforementioned properties thereof for a long period of time.

In addition, in accordance with the present invention, an emulsion of an organopolysiloxane exhibiting superior stability over time and superior storage stability can be easily obtained. Namely, the organopolysiloxane emulsion prepared by means of the preparation method of the present invention exhibits superior stability over time, and can be stored stably for a long period of time. In addition, the method for producing the emulsion of the present invention can easily carried out using a known emulsification apparatus.

### BRIEF DESCRIPTION OF THE DRAWINGS

FIG. 1 is an IR chart of a partially methylated polyglycerol graft type polydimethylsiloxane produced in “Evaluation of production of formaldehyde” which was subjected to heat deterioration at 50° C. for 3 weeks in air.

FIG. 2 is an IR chart of a mixture (concentration=80% by weight) of a partially methylated polyglycerol graft type polydimethylsiloxane produced in “Evaluation of production of formaldehyde” and a buffer solution at pH 6, which was subjected to heat deterioration at 50° C. for 3 weeks in air.

FIG. 3 is an IR chart of a polyoxyethylene graft type polydimethylsiloxane produced in “Evaluation of production of formaldehyde” which was subjected to heat deterioration at 50° C. for 3 weeks in air.

FIG. 4 is an IR chart of a mixture (concentration=80% by weight) of a polyoxyethylene graft type polydimethylsiloxane produced in “Evaluation of production of formaldehyde” and a buffer solution at pH 6, which was subjected to heat deterioration at 50° C. for 3 weeks in air.

### BEST MODES FOR CARRYING OUT THE INVENTION

The present invention relates to an emulsion containing (A) an organopolysiloxane, (B) a silicone-based surfactant represented by the aforementioned general formula (1), and (C) an aqueous medium. In the aforementioned silicone-based surfactant (B) used in the present invention, the terminal OH group is partially alkylated, and for this reason, hydrogen bonding between the aforementioned OH groups themselves is controlled, and low viscosity is exhibited. By emulsifying the aforementioned organopolysiloxane (A) having the siloxane skeleton in the aforementioned aqueous medium (C) by means of the aforementioned component (B) in the same manner as described above, an extremely stable emulsified condition can be formed. In addition, the emulsions of the present invention generally have low viscosity and handling thereof is easy. In addition, the aforementioned silicone-based surfactant (B) used in the present invention is difficult to be oxidized in air, as compared with a conventional polyether-modified (poly)siloxane and therefore, allergenic compounds such as formates, and aldehydes such as formaldehyde, are hardly produced during storage over time. For this reason, the emulsions obtained in accordance with the present invention exhibit increased environmental compatibility even if an after-treatment such as addition of an antioxidant, a hydrogenation treatment or the like is not carried out. Therefore, due to the aforementioned advantages, the emulsions of the present invention can be easily blended into cosmetics and the like, and are extremely useful as raw materials for cosmetics.

In addition, in the present invention, when an emulsion is produced by dispersing the organopolysiloxane in the aqueous medium or dispersing the aqueous medium in the organopolysiloxane due to effects of the aforementioned silicone-based surfactant (B) represented by the aforementioned general formula (1), the silicone-based surfactant is produced in situ in the organopolysiloxane to be emulsified, and then the aforementioned silicone-based surfactant is used as it is, together with the organopolysiloxane without combining a silicone-based surfactant already produced separately with the organopolysiloxane and then using them. Thereby, stability of the organopolysiloxane emulsion can be improved, as compared with the case in which, for example, a silicone-based surfactant is added to the organopolysiloxane from the outside.

Hereinafter, emulsions according to the present invention, methods for producing the same, and raw materials for cosmetics are described in detail with the components thereof.

The aforementioned organopolysiloxane (A) is an oil-based component, and any one can be used in the present invention. As the structure thereof, any one of straight chain, partially branched chain, branched chain and cyclic ones can be used. In general, the straight chain, partially branched chain or branched one is used. As examples of the organic group bonding to the silicon atom thereof, mention may be made of a substituted or non-substituted monovalent hydrocarbon group. More particularly, as examples of the substituted or non-substituted monovalent hydrocarbon group, mention may be made of, for example, saturated aliphatic hydrocarbon groups such as a methyl group, an ethyl group, a propyl group, a butyl group, a pentyl group, a hexyl group, a heptyl group, an octyl group, a decyl group, a dodecyl group, and the like; unsaturated aliphatic hydrocarbon groups such as a vinyl group, an allyl group, a hexenyl group and the like; saturated alicyclic hydrocarbon groups such as a cyclopentyl group, a cyclohexyl group and the like; aromatic hydrocarbon groups such as a phenyl group, a tolyl group, a naphthyl group and the like; and groups in which one or more hydrogen atoms bound to carbon atoms of the aforementioned groups are substituted with a halogen atom such as fluorine or the like, or an organic group containing an epoxy group, a glycidyl group, an acyl group, a carboxyl group, an amino group, a methacryl group, a mercapto group, or the like. The aforementioned organopolysiloxane (A) may contain a hydroxyl group or an alkoxy group bonding to the silicon atom.

As examples of the aforementioned organopolysiloxane (A), mention may be made of, for example,
α,ω-dihydroxypolydimethylsiloxane;
α,-hydroxy-ω-trimethylsiloxypolydimethylsiloxane;
α, ω-dimethoxypolydimethylsiloxane;
α-methoxy-ω-trimethylsiloxypolydimethylsiloxane;
α,ω-diethoxypolydimethylsiloxane;
α-ethoxy-ω-trimethylsiloxypolydimethylsiloxane;
α,ω-di(trimethylsiloxy)polydimethylsiloxane; crosslinked methylpolysiloxanes in which the terminal of the molecular chain is blocked with a silanol group, a methoxy group, an ethoxy group, or a trimethylsiloxy group; and organopolysiloxanes in which a part of the methyl groups in the aforementioned organopolysiloxanes is substituted with an ethyl group, a phenyl group, a vinyl group, a 3-aminopropyl group, an N-(2-aminoethyl)-3-aminopropyl group, a 3-methacryloxypropyl group, a 3-glycidoxypropyl group, or a 3-carboxypropyl group. The viscosity of the aforementioned organopolysiloxane (A) at 25° C. preferably ranges from 5 to 100,000 mPa·s, more preferably ranges from 10 to 10,000 mPa·s, further preferably ranges from 25 to 5, 000 mPa·s, and in particular, preferably ranges from 50 to 3,000 mPa·s.

As the aforementioned silicone-based surfactant (B), the following one can be used, which is represented by the following general formula (1):

R²₃SiO (R¹₂SiO)ₘ(R¹YSiO)ₙSiR²₃ (1)

wherein
each R¹ independently represents a hydrogen atom or a substituted or non-substituted monovalent hydrocarbon group;
each Y independently represents a group represented by the following general formula (2):

—CₐH₂ₐ(OC₂H₄)_{b}(OC₃H₆)_{c}—O—(B)_{d}-A (2)

wherein
A represents a terminal group represented by the following formula (3), (4) or (5):

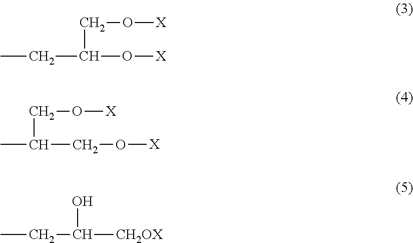

in each of the formulae, X represents a hydrogen atom or independently represents a substituted or non-substituted monovalent hydrocarbon group containing no aliphatic unsaturated bond, with not more than 20 carbon atoms; and at least one of the Xs is the aforementioned hydrocarbon group;
B represents a moiety represented by the following formula (6), (7), (8) or (9):

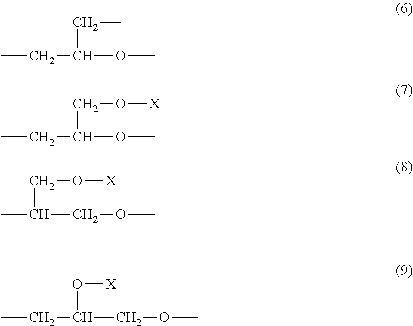

wherein
X is the same as described above;
(OC₂H₄) and (OC₃H₆) are arranged in any one of a random type, a block type, and an alternative type, or a mixed type thereof;
a ranges from 2 to 15, b ranges from 0 to 100, c ranges from 0 to 100, and b+c is preferably 100 or less; more preferably, a ranges from 2 to 10, b ranges from 5 to 50, c ranges from 0 to 50, and b+c is 50 or less; and further preferably, a ranges 2 to 7, b ranges from 10 to 30, c ranges from 0 to 30, and b+c is 30 or less; and d ranges from 0 to 500, preferably ranges from 1 to 500, more preferably ranges from 2 to 500, and further preferably ranges from 3 to 500, m ranges from 0 to 50 and n ranges from 0 to 20; preferably, m ranges from 0 to 30 and n ranges from 0 to 15; more preferably, m ranges from 0 to 10 and n ranges from 0 to 7; and further preferably, m ranges from 0 to 6 and n ranges from 0 to 3;
in particular, the case in which m ranges from 0 to 2 and n is 1, and the case in which m is 0 and n is 1 are preferable; and
R² represents R¹ or X, with the proviso that when n is 0, at least one R² represents X.

In the aforementioned terminal group A, all of the Xs present therein are not OH groups, and at least one X and preferably not less than 15% thereof are blocked by a hydrocarbon group. For this reason, hydrogen binding can be controlled. Therefore, the partially hydrocarbon group-blocked (poly) glycerol-modified polysiloxane has low viscosity, and superior handling properties are exhibited.

Preferably at least 20%, more preferably at least 30%, further more preferably at least 40%, further more preferably at least 50%, further more preferably at least 60% and further more preferably at least 70% of all the Xs present at the aforementioned terminal groups should be the aforementioned hydrocarbon group.

The amount of the aforementioned silicone-based surfactant (B) present in the emulsion is not particularly limited. The amount preferably ranges from 0.01 to 50 parts by weight and more preferably ranges from 0.1 to 30 parts by weight with respect to 100 parts by weight of the aforementioned organopolysiloxane (A).

As an example of the method for producing the aforementioned silicone-based surfactant (B), mention may be made of, for example, a method in which a silicon atom-bonding hydrogen atom-containing siloxane and a terminal double bond-containing compound are subjected to a hydrosilylation reaction in the presence of a catalyst for use in a hydrosilylation reaction, as a representative example.

In the aforementioned example, the silicon atom-bonding hydrogen atom-containing siloxane can be represented by the following general formula (1′):

R²₃SiO(R¹₂SiO)ₘ(R¹HSiO)ₙSiR²₃ (1′)

wherein
R¹, R², m and n are as described above.

As examples of silicon atom-bonding hydrogen atom-containing siloxanes of the general formula (1′), mention may be made of, for example, 1,2-dihydrogen-1,1,2,2-tetramethyldisiloxane, 1-hydrogen-1,1,2,2,2-pentamethyldisiloxane, 2-hydrogen-1,1,1,2,3,3,3-heptamethyltrisiloxane, 1,3-dihydrogen-1,1,2,2,3,3-hexamethyltrisiloxane, 1-hydrogen-1,1,2,2,3,3,3-heptamethyltrisiloxane, 1-hydrogen-1,1,2,2,3,3,4,4,4-nonamethyltetrasiloxane, 3-hydrogen-1,1,1,2,2,3,4,4,4-nonamethyltetrasiloxane, and the like. 2-hydrogen-1,1,1,2,3,3,3-heptamethyltrisiloxane is, in particular, preferable.

In addition, in the aforementioned examples, the terminal double bond-containing compound can be represented by the following general formula (2′):

CH₂═CH—C_{a′}H_{2a′}(OC₂H₄)_{b}(OC₃H₆)_{c}—O—(B)_{d}-A (2 ′)

wherein
A, B, b, c and d are as described above; a′ ranges from 0 to 13, preferably ranges from 0 to 8, and more preferably ranges from 1 to 3.

The aforementioned terminal double bond-containing compound can be obtained by, for example, subjecting glycidyl ether obtained by replacing the hydrogen atom in the hydroxyl group of glycidol with the hydrocarbon group for forming the aforementioned X group, and optionally together with glycidol, if necessary, to a ring-opening (co)polymerization in the presence of an acid or basic catalyst using an aliphatic unsaturated bond-containing alcohol or carboxylic acid such as ethylene glycol monoallyl ether or the like as an initiator. The ring-opening (co)polymerization can be carried out in accordance with a conventional method. When a mixture of the glycidyl ether and glycidol are copolymerized, one corresponding to a random copolymer can be obtained. On the other hand, when one is polymerized and then the other is added to polymerize these, one corresponding to a block copolymer can be obtained. Two or more types of glycidyl ethers can also be used to copolymerize with glycidol.

In addition, the aforementioned terminal double bond-containing compound can also be produced by means of a so-called Williamson ether synthesis reaction, which comprises subjecting glycidol to a ring-opening polymerization in the presence of an acid or basic catalyst using the aforementioned aliphatic unsaturated bond-containing alcohol or carboxylic acid as an initiator, subsequently adding a specified amount of an alkali hydroxide to form an alkali-alcholated terminal of a molecular chain, and subsequently reacting with a halogenated hydrocarbon to partially replace hydrogen atoms in the hydroxyl groups with hydrocarbon groups.

As examples of the acid polymerization catalyst, mention may be made of Lewis acids such as BF₃.OEt₂, HPF₆.OEt₂, TiCl₄, SnCl₄, sulfuric acid, PhCOSbF₆, perchloric acid, fluorosulfuric acid, trifluoroacetic acid, trifluoromethanesulfonic acid and the like, wherein Et represents an ethyl group; and Ph represents a phenyl group. As examples of basic polymerization catalysts, mention may be made of a metal hydroxide such as LiOH, NaOH, KOH, CsOH or the like; an alkali metal such as Li, Na, K, Cs or the like or mercury amalgam thereof; a metal alcholate represented by the following general formula: ROM¹, wherein R=alkyl group, and preferably an alkyl group having 1 to 4 carbon atoms, and M¹=alkali metal; a metal hydride of which the metal is an alkali metal or an alkaline earth metal; an organometal compound such as n-butyl lithium, t-butyl lithium, potassium pentadienyl, potassium naphthalene, Grignard reagent or the like; and the like. Among these, the alkali metal, metal hydroxide, metal alcholate or organometal compound is preferable due to high activity. In particular, K, KOH, CsOH, potassium hydride, potassium methoxide, potassium isopropoxide, or potassium t-butoxide is, in particular, preferable as a catalyst having both convenience and increased activity. The amount of the catalyst preferably ranges from 0.01 to 2 molar equivalents, more preferably ranges from 0.03 to 1.0 molar equivalents, and in particular, preferably ranges from 0.05 to 0.8 molar equivalents with respect to one molar equivalent of the functional group.

A solvent may or may not be used. When the reaction system has an extremely increased viscosity or is in the form of a solid or a non-uniform slurry mixture in accordance with the catalyst type, the amount of the catalyst, or the blending amount of glycidol, a suitable solvent is used and a polymerization reaction can be carried out therein.

The polymerization temperature may be suitably determined in accordance with polymerization activity of the catalyst used, concentration of the functional group thereof, and the like, and ranges from −78 to 220° C., and more preferably ranges from −30 to 150° C.

In the chain of the aforementioned terminal double bond-containing compound, a small amount of an ethyleneoxy group and/or a propyleneoxy group may be present. The aforementioned groups are unstable with respect to oxidation and are easily decomposed to give a carbonyl functional decomposed product. For this reason, the amount of the aforementioned groups is preferably not more than 0.5 molar equivalents and more preferably not more than 0.2 molar equivalents with respect to one molar equivalent of a polyglycerol group. They can be easily produced by adding a specified amount of ethylene oxide and/or propylene oxide in the aforementioned polymerization reaction to perform copolymerization.

In the aforementioned examples, as examples of the catalyst for use in hydrosilylation reaction, mention may be made of, for example, platinum-based catalysts, rhodium-based catalysts, and palladium-based catalysts. Among these, the platinum-based catalysts are preferable since addition polymerization is remarkably accelerated. In particular, platinum microparticles, platinum-supported silica micropowders, platinum-supported activated-carbon, chloroplatinic acid, alcohol solution of chloroplatinic acid, platinum-alkenylsiloxane complex, platinum-olefin complex, and platinum-carbonyl complex can be mentioned as examples, and in particular, platinum-alkenylsiloxane complex is preferable. As examples of the aforementioned alkenylsiloxane, 1,3-divinyl-1,1,3,3-tetramethyldisiloxane; 1,3,5,7-tetramethyl-1,3,5,7-tetravinylcyclotetrasiloxane, alkenylsiloxanes in which a part of the methyl groups of the aforementioned alkenylsiloxanes is substituted with an ethyl group, a phenyl group or the like, and alkenylsiloxanes in which a part of the vinyl groups of the aforementioned alkenylsiloxanes is substituted with an allyl group, a hexenyl group or the like. Among these, 1,3-divinyl-1,1,3,3-tetramethyldisiloxane is preferable since good stability as a complex is exhibited. In addition, in order to improve stability of the aforementioned platinum-alkenylsiloxane complex, an alkenylsiloxane such as 1,3-divinyl-1,1,3,3-tetramethyldisiloxane, 1,3-diallyl-1,1,3,3-tetramethyldisiloxane, 1,3-divinyl-1,3-dimethyl-1,3-diphenyldisiloxane, 1,3-divinyl-1,1,3,3-tetraphenyldisiloxane, 1,3,5,7-tetramethyl-1,3,5,7-tetravinylcyclotetrasiloxane or the like; or an organosiloxane oligomer such as dimethylsiloxane oligomer, or the like is preferably added thereto to form a liquid catalyst. Among these, the alkenylsiloxane is preferable.

The emulsion of the present invention can be produced by emulsifying the aforementioned organopolysiloxane (A) together with the aforementioned silicone-based surfactant (B) in the aforementioned aqueous medium (C) by means of a known emulsifying means with a mechanical force. The forms of the emulsions may be any one of oil-in-water emulsions, and water-in-oil emulsions. The form of an oil-in-water emulsion is preferable.

As the aforementioned aqueous medium (C), water or a mixture between water and an organic solvent miscible with water at room temperature (25° C.) (water-miscible organic solvent) can be used. Suitably, not less than 75% by mass of the aforementioned aqueous medium (C) is preferably water, not less than 90% by mass of the aforementioned aqueous medium (C) is preferably water, and the aforementioned aqueous medium (C) is most preferably substantially water. In the case of applying the emulsion according to the present invention to a cosmetic raw material or the like, the water is preferably pure. As examples thereof, mention may be made of purified water, ion-exchanged water, and naturally or artificially heat-treated or sterilization-treated mineral water.

As examples of the organic solvents miscible with water at room temperature (25° C.), mention may be made of, for example, monoalcohols having 2 to 6 carbon atoms such as ethanol, isopropanol, and the like; polyols having 2 to 20 carbon atoms, preferably having 2 to 10 carbon atoms, and more preferably having 2 to 6 carbon atoms such as glycerol, propylene glycol, butylene glycol, pentylene glycol, hexylene glycol, dipropylene glycol, and diethylene glycol; glycol ethers, and in particular, those having 3 to 16 carbon atoms, such as (C₁-C₄) alkyl ethers of mono-, di- or tripropylene glycol, and (C₁-C₄) alkyl ethers of mono-, di- or triethylene glycol; and mixtures thereof.

The amount of the aqueous medium (C) is not particularly limited. The amount can range from 10 to 10,000 parts by weight, preferably range from 100 to 10,000 parts by weight, and more preferably range from 300 to 10, 000 parts by weight with respect to 100 parts by weight of the aforementioned organopolysiloxane (A).

The emulsifying means is not particularly limited. For example, a known stirring/mixing apparatus or emulsifier such as a homomixer, paddle mixer, Henschel mixer, homodisper, colloid mixer, propeller stirrer, homogenizer, in-line type continuous emulsifier, ultrasonic emulsifier, vacuum type kneader, colloid mill, combination mixer or the like can be appropriately used.

More particularly, the emulsion of the present invention can be produced by means of a method in which the aforementioned organopolysiloxane (A) and silicone-based surfactant (B) are emulsified and dispersed in the aforementioned aqueous medium (C) by means of the aforementioned emulsification means. More preferably, the aforementioned component (B) is synthesized in situ in the aforementioned component (A) as described below, and the obtained mixture is emulsified to produce an organopolysiloxane emulsion. This is, in particular, preferable in view of stability of an emulsion.

In the present invention, a mixture between the aforementioned organopolysiloxane (A) and the aforementioned silicone-based surfactant (B) synthesized in situ in the aforementioned organopolysiloxane (A) is preferably emulsified to produce an emulsion. Emulsification can be carried out by combining the aforementioned mixture with an aqueous medium. The forms of the emulsions may be any one of oil-in-water emulsions, or water-in-oil emulsions. The form of an oil-in-water emulsion is preferable.

In the present invention, silicone-based surfactants other than the aforementioned silicone-based surfactant (B) and/or other surfactants such as surfactants having higher hydrocarbon groups and the like can be added to the emulsion. As examples of the aforementioned other surfactants, mention may be made of silicone-based surfactants such as polyether-modified (poly)siloxanes other than those represented by the aforementioned general formula (1), polyglycerol-modified (poly)siloxanes other than those represented by the aforementioned general formula (3), poly(glycidyl ether)-modified (poly)siloxanes other than those represented by the aforementioned general formula (3), poly (glycidyl ether)-polyglycerol-modified (poly)siloxanes other than those represented by the aforementioned general formula (3), and the like; anionic surfactants such as hexylbenzenesulfonic acid, octylbenzenesulfonic acid, decylbenzenesulfonic acid, dodecylbenzenesulfonic acid, cetylbenzenesulfonic acid, myristylbenzenesulfonic acid, sodium salts thereof, and the like; cationic surfactants such as octyl trimethylammonium hydroxide, dodecyl trimethylammonium hydroxide, hexadecyl trimethylammonium hydroxide, octyldimethylbenzylammonium hydroxide, decyl dimethylbenzylammonium hydroxide, dioctadecyl dimethylammonium hydroxide, beef tallow trimethylammonium hydroxide, coconut oil trimethylammonium hydroxide and the like; nonionic surfactants such as polyoxyalkylene alkyl ether, polyoxyalkylene alkylphenol, polyoxyalkylene alkyl ester, polyoxyalkylene sorbitan ester, polyethylene glycol, polypropylene glycol, ethylene oxide adducts of diethylene glycol trimethylnonanol or polyester-based nonionic surfactants; and mixtures of two or more types of the aforementioned surfactants. The addition amount thereof is not limited, but preferably ranges from 0.01 to 50 parts by weight, and more preferably ranges from 0.1 to 30 parts by weight, with respect to 100 parts by weight of the aforementioned organopolysiloxane (A).

The timing of adding the aforementioned other surfactants is not particularly limited. The addition is preferably carried out after the aforementioned silicon-based surfactant (B) is synthesized in the aforementioned organopolysiloxane (A) or before the aforementioned silicon-based surfactant (B) is synthesized in the aforementioned organopolysiloxane (A).

The aforementioned organopolysiloxane (A) to be emulsified in the present invention may be combined with a non-silicone oil. The aforementioned non-silicone oils are not particularly limited, and any types of oils can be used. The origins from the non-silicone oils are not particularly limited, and the oils may be in the form of a solid, a semi-solid, or a liquid, and may be non-volatile, semi-volatile, or volatile, as long as they are hydrophobic. More particularly, as examples thereof, mention may be made of hydrocarbon oils and waxes, animal or vegetable oils, higher alcohols, ester oils and the like. The oils may be used in one type thereof alone or in combination with two or more types thereof.

As examples of hydrocarbon oils and waxes, mention may be made of, for example, ozocerite, squalane, squalene, ceresin, paraffin, paraffin wax, liquid paraffin, pristane, polyisobutylene, polybutene, microcrystalline wax, vaseline, and the like. They may be used in combination with two or more types thereof.

As examples of animal or vegetable fats and oils, mention may be made of, for example, avocado oil, linseed oil, almond oil, ibota wax, perilla oil, olive oil, cacao butter, kapok wax, kaya oil, carnauba wax, liver oil, candelilla wax, beef tallow, neat's-foot oil, beef bone fat, hydrogenated beef tallow, apricot kernel oil, spermaceti wax, hydrogenated oil, wheat germ oil, sesame oil, rice germ oil, rice bran oil, sugar cane wax, sasanqua oil, safflower oil, shear butter, Chinese tung oil, cinnamon oil, jojoba wax, shellac wax, turtle oil, soybean oil, tea seed oil, camellia oil, evening primrose oil, corn oil, lard, rapeseed oil, Japanese tung oil, rice bran wax, germ oil, horse fat, persic oil, palm oil, palm kernel oil, castor oil, hydrogenated castor oil, castor oil fatty acid methyl ester, sunflower oil, grape oil, bayberry wax, jojoba oil, macadamia nut oil, beeswax, mink oil, cottonseed oil, cotton wax, Japanese wax, Japanese wax kernel oil, montan wax, coconut oil, hydrogenated coconut oil, tri-coconut oil fatty acid glyceride, mutton tallow, peanut oil, lanolin, liquid lanolin, reduced lanolin, lanolin alcohol, hard lanolin, lanolin acetate, lanolin fatty acid isopropyl ester, hexyl laurate, POE lanolin alcohol ether, POE lanolin alcohol acetate, lanolin fatty acid polyethylene glycol, POE hydrogenated lanolin alcohol ether, egg yolk oil, and the like. They may be used in combination with two or more types thereof.

As examples of higher alcohols, mention may be made of, for example, lauryl alcohol, myristyl alcohol, palmityl alcohol, stearyl alcohol, behenyl alcohol, hexadecyl alcohol, oleyl alcohol, isostearyl alcohol, hexyldodecanol, octyldodecanol, cetostearyl alcohol, 2-decyltetradecinol, cholesterol, phytosterol, POE cholesterol ether, monostearyl glycerol ether (batyl alcohol), monooleyl glyceryl ether (selachyl alcohol) and the like. They may be used in combination with two or more types thereof.

As examples of ester oils, mention may be made of, for example, diisobutyl adipate, 2-hexyldecyl adipate, di-2-heptylundecyl adipate, N-alkylglycol monoisostearate, isocetyl isostearate, trimethylolpropane triisostearate, ethylene glycol di-2-ethylhexanoate, cetyl 2-ethylhexanoate, trimethylolpropane tri-2-ethylhexanoate, pentaerythritol tetra-2-ethylhexanoate, cetyl octanoate, octyldodecyl gum ester, oleyl oleate, octyldodecyl oleate, decyl oleate, isononyl isononanoate, neopentyl glycol dicaprate, triethyl citrate, 2-ethylhexyl succinate, amyl acetate, ethyl acetate, butyl acetate, isocetyl stearate, butyl stearate, diisopropyl sebacate, di-2-ethylhexyl sebacate, cetyl lactate, myristyl lactate, isopropyl palmitate, 2-ethylhexyl palmitate, 2-hexyldecyl palmitate, 2-heptylundecyl palmitate, cholesteryl 12-hydroxystearate, dipentaerythritol fatty acid ester, isopropyl myristate, 2- ethylhexyl myristate, octyldodecyl myristate, 2-hexyldecyl myristate, myristyl myristate, hexyldecyl dimethyloctanoate, ethyl laurate, hexyl laurate, 2-octyldodecyl N-lauroyl-L-glutamate, diisostearyl malate, and the like. As examples of glyceride oils, mention may be made of acetoglyceryl, glyceryl triisooctanoate, glyceryl triisostearate, glyceryl triisopalmitate, glyceryl tri(caprylate/caprate), glyceryl monostearate, glyceryl di-2-heptylundecanoate, glyceryl trimyristate, diglyceryl myristate isostearate, and the like. They can be used in combination with two or more types thereof.

In the present invention, known other components can be added or blended as additives within a range which does not impair the objectives of the present invention, before emulsification or after emulsification. As examples of the aforementioned additives, mention may be made of hydrolysable organosilanes, silicas, pH adjustors, preservatives, fungicides, anti-corrosion agents, and thickeners. The aforementioned components may be used alone or in combination with plural types.

In addition, in the present invention, before the aforementioned silicone-based surfactant (B) is synthesized, a part of the aforementioned aqueous medium can be preliminarily mixed in the aforementioned organopolysiloxane (A). The amount of the aqueous medium to be preliminarily mixed is not particularly limited. The amount preferably ranges from 0.01 to 50 parts by weight and more preferably ranges from 0.01 to 20 parts by weight with respect to 100 parts by weight of the organopolysiloxane (A).

### EXAMPLES

Hereinafter, the present invention is described in detail with reference to examples. It should be understood that the present invention is not limited to the examples.

### Reference Example 1

Ethylene glycol monoallyl ether, in an amount of 1.88 g (18.4 mmol), and potassium t-butoxide, in an amount of 0.10 g (0.88 mmol), were mixed and the mixture was heated at 105° C. under a nitrogen atmosphere. A mixture of 10.9 g (147.2 mmol) of glycidol and 6.5 g (73.6 mmol) of glycidyl methyl ether was slowly added dropwise thereto over 3.5 hours at 115 to 120° C. (molar ratio of ethylene glycol monoallyl ether:glycidol:glycidyl methyl ether=1:8:4). After completion of the dropwise addition, the mixture was heated and stirred for 3 hours at 120° C. The mixture was cooled to room temperature, and 0.06 g of acetic acid was added thereto to stop the polymerization. Toluene in an amount of 10 g was added thereto, and KYOWADO 500 SN, which is a hydrotalcite-based absorbent manufactured by Kyowa Chemical Industry Co., Ltd., was added thereto, and the mixture was stirred for 2 hours. After the mixture was filtered, the materials with low boiling points were removed from the filtrate by heating under reduced pressure. Thereby, 18.8 g (yield=98%) of a transparent liquid polymer was obtained. The polymer was slightly heated, and thereby, it could be easily taken out from the reactor. The number average molecular weight thereof on the basis of standard polystyrene, measured by means of gel permeation chromatography (GPC) by a refractive index detector with chloroform as a solvent was 249 and the degree of dispersion was 1.785. In addition, from the results of ¹³C-nuclear magnetic resonance (¹³C-NMR) analysis, the present polymer was an allyloxyethoxy-terminal methyl polyglycerol, and the molar ratio of carbinol group:methoxy group was 69:21. In addition, the signal of a —CH₂—CH(—CH₂O—)O— group showing a branched structure was observed at 78 to 81 ppm.

### Reference Examples 2 to 7

In the same manner as described in Reference Example 1, a polymerization reaction was carried out with the composition shown in a table described below, and the corresponding allyloxyethoxy-terminal methyl polyglycerol was obtained. The results are shown in Table 1 and Table 2.

TABLE 1

| | Reference | Reference | Reference | Reference |
|---|---|---|---|---|
| | Example 2 | Example 3 | Example 4 | Example 5 |
| Ethylene glycol | 1.88 | 1.88 | 1.88 | 1.88 |
| monoallyl ether (A) (g) | | | | |
| Potassium t-butoxide | 0.1 | 0.1 | 0.1 | 0.1 |
| (g) | | | | |
| Glycidyl methyl ether | 3.24 | 9.72 | 12.96 | 16.21 |
| (B) (g) | | | | |
| Glycidol (C) (g) | 13.63 | 8.17 | 5.45 | 2.73 |
| Number average | —* | 470 | 1123 | 1328 |
| molecular weight | | | | |
| Degree of dispersion | —* | 1.721 | 1.378 | 1.388 |
| Molar ratio (A:B:C) | 1:2:10 | 1:6:6 | 1:8:4 | 1:10:2 |
| Molar ratio (OH group: | 85:15 | 54:46 | 38:62 | 23:77 |
| CH₃ group) | | | | |

*insoluble in chloroform

TABLE 2

| | Reference | Reference |
|---|---|---|
| | Example 6 | Example 7 |
| Ethylene glycol monoallyl ether | 3.76 | 0.94 |
| (A) (g) | | |
| Potassium t-butoxide (g) | 0.1 | 0.1 |
| Glycidyl methyl ether (B) (g) | 12.96 | 12.96 |
| Glycidol (C) (g) | 5.45 | 5.45 |
| Number average molecular weight | 753 | 1430 |
| Degree of dispersion | 1.397 | 1.445 |
| Molar ratio (A:B:C) | 1:4:2 | 1:16:8 |
| Molar ratio (OH group:CH₃ group) | 43:57 | 36:64 |

### Reference Example 8

Ethylene glycol monoallyl ether, in an amount of 1.88 g (18.4 mmol), and potassium t-butoxide, in an amount of 0.10 g (0.88 mmol), were mixed, and the mixture was heated at 120° C. under a nitrogen atmosphere.

Glycidol, in an amount of 5.45 g (73.6 mmol) , was slowly added dropwise thereto over 1.5 hours at 115 to 120° C. After completion of the dropwise addition, the mixture was heated and stirred for 2 hours at 120° C. to complete polymerization. Subsequently, 12.96 g (147.2 mmol) of glycidyl methyl ether was added thereto. Subsequently, the mixture was heated and stirred for 3 hours at 120 to 130° C. to complete block copolymerization (molar ratio of ethylene glycol monoallyl ether:glycidol:glycidyl methyl ether=1:4:8). The mixture was cooled to room temperature, and 0.06 g of acetic acid was added thereto, to stop the polymerization. Toluene in an amount of 10 g was added thereto, and KYOWADO 500 SN, which is a hydrotalcite-based absorbent manufactured by Kyowa Chemical Industry Co., Ltd., was added thereto, and the mixture was stirred for 2 hours. After the mixture was filtered, the materials with low boiling points were removed from the filtrate by heating under reduced pressure. Thereby, 19.9 g (yield=98%) of a transparent liquid polymer was obtained. The number average molecular weight thereof on the basis of standard polystyrene, measured by means of gel permeation chromatography (GPC) by a refractive index detector with chloroform as the solvent was 1,412 and the degree of dispersion was 1.271. In addition, from the results of ¹³C-nuclear magnetic resonance (¹³C-NMR) analysis, the present polymer was an allyloxyethoxy-terminal methyl polyglycerol, and the molar ratio of carbinol group:methoxy group was 38:62. In addition, the signal of a —CH₂—CH (—CH₂O—)O— group showing a branched structure was observed at 78 to 81 ppm.

The reaction scheme in Reference Example 8 is generally described as follows:

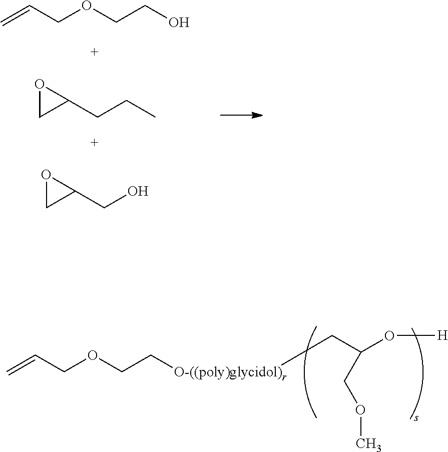

### Reference Example 9 and Reference Example 10

The corresponding allyloxyethoxy-terminal methyl polyglycerols were obtained by carrying out a polymerization reaction in the same manner as described in Reference Examples 1 to 7 with the compositions described below using glycidyl ethyl ether instead of glycidyl methyl ether. The results are shown in Table 3.

TABLE 3

| | Reference | Reference |
|---|---|---|
| | Example 9 | Example 10 |
| Ethylene glycol monoallyl ether | 1.88 | 1.17 |
| (A) (g) | | |
| Potassium t-butoxide (g) | 0.1 | 0.1 |
| Glycidyl methyl ether (B) (g) | 7.52 | 7.00 |
| Glycidol (C) (g) | 10.90 | 5.07 |
| Number average molecular weight | 339 | — |
| Degree of dispersion | 1.875 | — |
| Molar ratio (A:B:C) | 1:4:8 | 1:6:6 |
| Molar ratio (OH group:CH₃ group) | 38:62 | 54:46 |

Evaluation of Stability Over Time

Stability over time of the emulsions obtained in the present invention were evaluated. In the below description, “parts” indicates parts by weight.

The viscosity of the organopolysiloxane, as well as, the average particle size of the emulsion and stability over time of the emulsion were measured in accordance with the following methods.

Viscosity of Organopolysiloxane

The viscosity of each organopolysiloxane was measured at 25° C. by means of a rotational viscometer (Rotor No.3).

Average Particle Size of Emulsion

The average particle size was measured by means of a laser scattering type submicron particle analyzer (COULTER N4 model, manufactured by Coulter Electronics Co., Ltd.).

Stability Over Time of Emulsion

The produced emulsion, in an amount of 100 g, was placed in a glass bottle with a volume of 100 cc, followed by allowing to stand at 25° C. Visual observation thereof was periodically carried out. In accordance with the period until separation of an oil phase from the emulsion was observed, evaluation was carried out with 6 stages of the following 0 to 5.
0: No emulsion was formed.
1: The oil phase was separated within one week.
2: The oil phase was separated after one week, but within one month.
3: The oil phase was not separated after not less than one month.
4: The oil phase was not separated after not less than two months.
5: The oil phase was not separated after not less than four months.

### Example 1

1.45 parts of 2-hydrogen-1,1,1,2,3,3,3-heptamethyltrisiloxane and 10.2 parts of the allyloxyethoxy-terminal methyl polyglycerol (12 mol) obtained in Reference Example 1 were added to 50 parts of an OH-terminal polydimethylsilicone (viscosity=about 100 mPa·s), followed by mixing them until a uniform mixture was obtained. After the mixture was heated to 70° C., 0.04 parts of a solution of a platinum catalyst was added thereto to react them for 15 minutes at 70° C. Thereby, a mixture of the OH-terminal polydimethylsilicone and methyl polyglycerol-modified silicone was obtained. After the mixture was cooled, 15 parts of water was added thereto and emulsification was carried out for 15 minutes at about 2,500 rpm by means of a T. K homodisper (manufactured by Tokushu Kika Kogyo Co., Ltd.). Subsequently, 23.31 parts of water was added thereto, and diluted. Thereby, an emulsion was obtained. The particle size of the obtained emulsion was 278 nm. The emulsion was stable for 6 months.

### Example 2

1.5 parts of 2-hydrogen-1,1,1,2,3,3,3-heptamethyltrasiloxane and 10.2 parts of the allyloxyethoxy-terminal methyl polyglycerol (8 mol) obtained in Reference Example 1-polyglycerol (4 mol) were added to 50 parts of an OH-terminal polydimethylsilicone (viscosity=about 100 mPa·s), followed by mixing them until a uniform mixture was obtained. After the mixture was heated to 70° C., 0.04 parts of a solution of a platinum catalyst was added thereto to react them for 15 minutes at 70° C. Thereby, a mixture of the OH-terminal polydimethylsilicone and methyl polyglycerol-modified silicone was obtained. After the mixture was cooled, 20 parts of water was added thereto and emulsification was carried out for 15 minutes at about 2,500 rpm by means of a T. K homodisper (manufactured by Tokushu Kika Kogyo Co., Ltd.). Subsequently, 18.26 parts of water was added thereto, and diluted. Thereby, an emulsion was obtained. The particle size of the obtained emulsion was 584 nm. The emulsion was stable for 3 months.

### Example 3

1.04 parts of 2-hydrogen-1,1,1,2,3,3,3-heptamethyltrisiloxane and 14.0 parts of the allyloxyethoxy-terminal methyl polyglycerol (25 mol) obtained in Reference Example 1 were stirred. After 0.04 parts of a platinum catalyst was added thereto, the mixture was heated to 70° C. and was maintained for 15 minutes. Thereby, a methyl polyglycerol-modified silicone was obtained. This silicone was used as Emulsifier A. 5 parts of Emulsifier A was added to 75 parts of water, followed by mixing them until a uniform mixture was obtained. 20 parts of an OH-terminal polydimethylsiloxane (100 mPa·s) was added thereto, and the mixture was stirred. The mixture was treated twice with 500 kg/cm² by means of a nanomizer. Thereby, an emulsion was obtained. In the obtained emulsion, oil separation or water separation was not observed, and the particle size thereof was 184 nm. The obtained emulsion was stable for one month.

### Example 4

1.9 parts of 2-hydrogen-1,1,1,2,3,3,3-heptamethyltrisiloxane and 7.0 parts of the allyloxyethoxy-terminal methyl polyglycerol (6 mol) obtained in Reference Example 1 were stirred. After 0.02 parts of a platinum catalyst was added thereto, the mixture was heated to 70° C. and was maintained for 15 minutes. Thereby, a methyl polyglycerol-modified silicone was obtained. This silicone was used as Emulsifier B. 5 parts of Emulsifier B was added to 75 parts of water, followed by mixing them until a uniform mixture was obtained. 20 parts of an OH-terminal polydimethylsiloxane (100 mPa·s) was added thereto, and the mixture was stirred. The mixture was treated twice with 500 kg/cm² by means of a nanomizer. Thereby, an emulsion was obtained. In the obtained emulsion, oil separation or water separation was not observed, and the particle size thereof was 554 nm. The obtained emulsion was stable for one month.

### Example 5

2.5 parts of Emulsifier A of Example 3 and 2.5 parts of Emulsifier B of Example 4 were added to 75 parts of water, followed by mixing them until a uniform mixture was obtained. 20 parts of an OH-terminal polydimethylsiloxane (viscosity=about 100 mPa·s) was added thereto, and the mixture was stirred. The mixture was treated twice with 500 kg/cm² by means of a nanomizer. Thereby, an emulsion was obtained. In the obtained emulsion, oil separation or water separation was not observed, and the particle size thereof was 190 nm. The obtained emulsion was stable for one month.

Evaluation for Producibility of Formaldehyde

The ability of producing formaldehyde of the silicone-based surfactant used in the present invention was evaluated as compared with a polyether-modified silicone.

In a flask with four necks, equipped with a stirrer, 7.5 g (12.4 mmol) of the allyloxyethoxy-terminal methyl polyglycerol synthesized in Reference Example 6, 2.97 g (SiH: 10.37 mmol) of a copolymer of polydimethylsiloxane and polymethylhydrogensiloxane represented by the following formula (I):

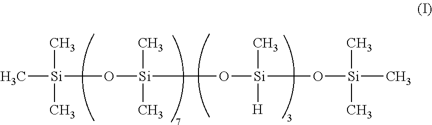

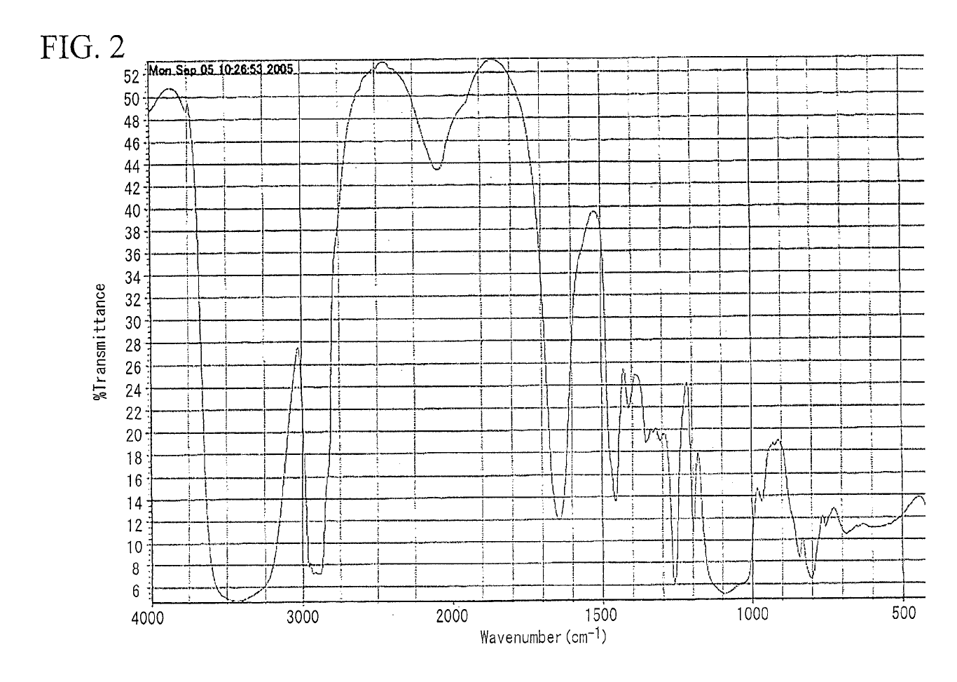

and 4 g of toluene were mixed, and a complex between platinum and 1,3-divinyl-tetramethyldisiloxane was mixed therewith so that the amount of the metal platinum was 5 ppm. The mixture was stirred for 3 hours at 80° C. As a result of infrared (IR) absorption analysis thereof by sampling, the characteristic absorption of the silicon atom-bonding hydrogen atom disappeared, and the reaction was completed. The materials with low boiling points were removed by heating and distilling under reduced pressure. Thereby, a transparent pale yellow polymer was obtained. As a result of ²⁹Si and ¹³C nuclear magnetic resonance (NMR) analysis of the polymer (see FIG. 2), it can be seen that the polymer was a methyl polyglycerol-modified silicone. The number average molecular weight thereof on the basis of standard polystyrene, measured by means of gel permeation chromatography (GPC) by a refractive index detector with chloroform as the solvent was 1,303 and the degree of dispersion was 2.445. The obtained polysiloxane exhibited fluidity even at room temperature, and by slightly heating, the polysiloxane could be easily taken out from the reactor. In addition, the polysiloxane exhibited complete compatibility with water, and a transparent aqueous solution thereof could be obtained. As a result of measuring the cloud point after a 0.5% by weight aqueous solution was prepared and heated, the cloud point was 25° C.

The aforementioned methyl polyglycerol-modified silicone, in an amount of 2 g, or a polyether-modified silicone having a polysiloxane content index and a value of the measured molecular weight which were close to those of the methyl polyglycerol-modified silicone, having a structure shown by the following formula (II):

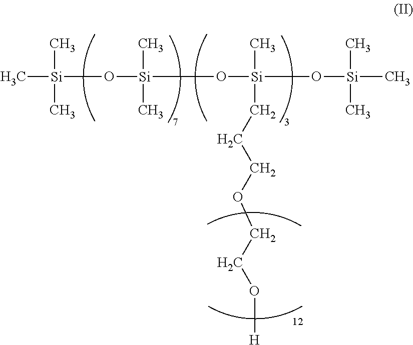

in an amount of 2 g, as a single material, as well as, a solution obtained by mixing each of the aforementioned methyl polyglycerol-modified silicone or polyether-modified silicone with a buffer solution at pH 6 so that the concentration of the aforementioned methyl polyglycerol-modified silicone or polyether-modified silicone was 80% by weight, in an amount of 2 g, were independently placed in a glass bottle with a volume of 30 cc and sealed under an air atmosphere, followed by subjecting them to a deterioration treatment by heating for 3 weeks in an oven at 50° C.

After the bottle was returned to room temperature, the presence of formaldehyde was checked by using a Formaldehyde Test Strip (TR) manufactured by Kanto Chemical Co., Inc., which is a test paper for selectively detecting formaldehyde. As a result, in both cases of the single material of the polyether-modified silicone represented by the aforementioned formula (II) and the mixture thereof with the buffer solution at pH 6, yellowing was observed, and formaldehyde was detected. On the other hand, in both cases of the single material of the aforementioned methyl polyglycerol-modified silicone and the mixture thereof with the buffer solution at pH 6, changing in color was not observed, and it could not be confirmed that formaldehyde was produced.

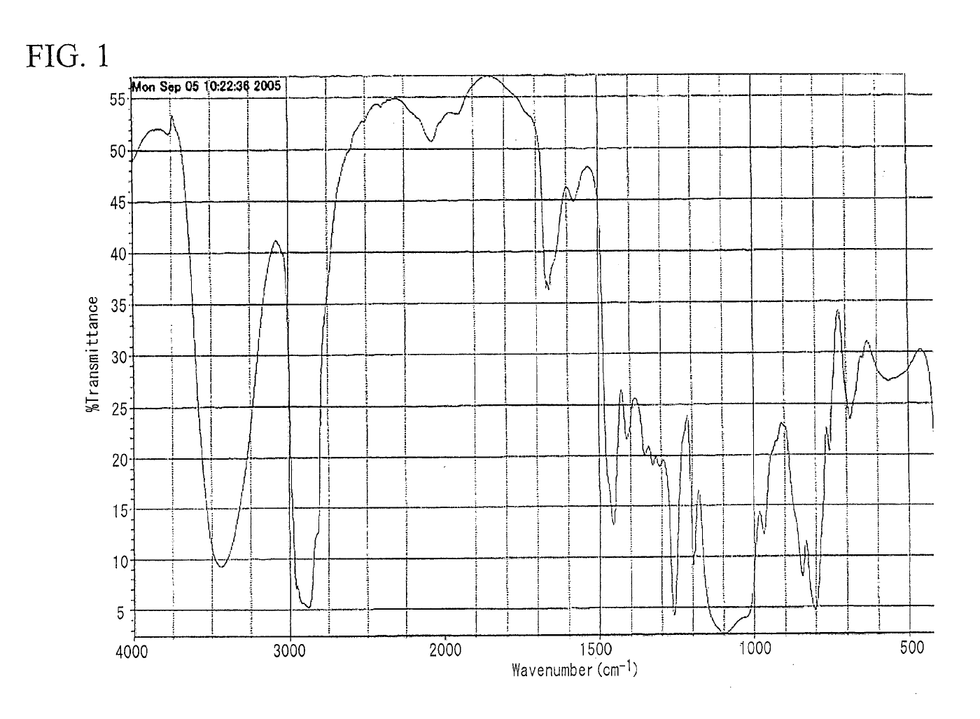

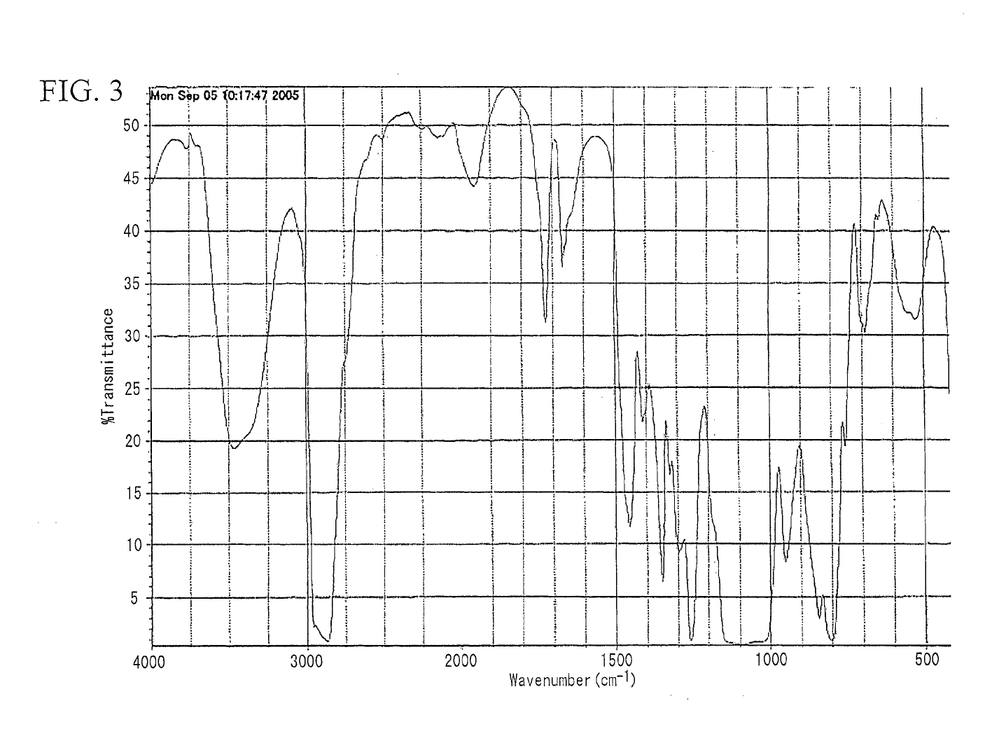

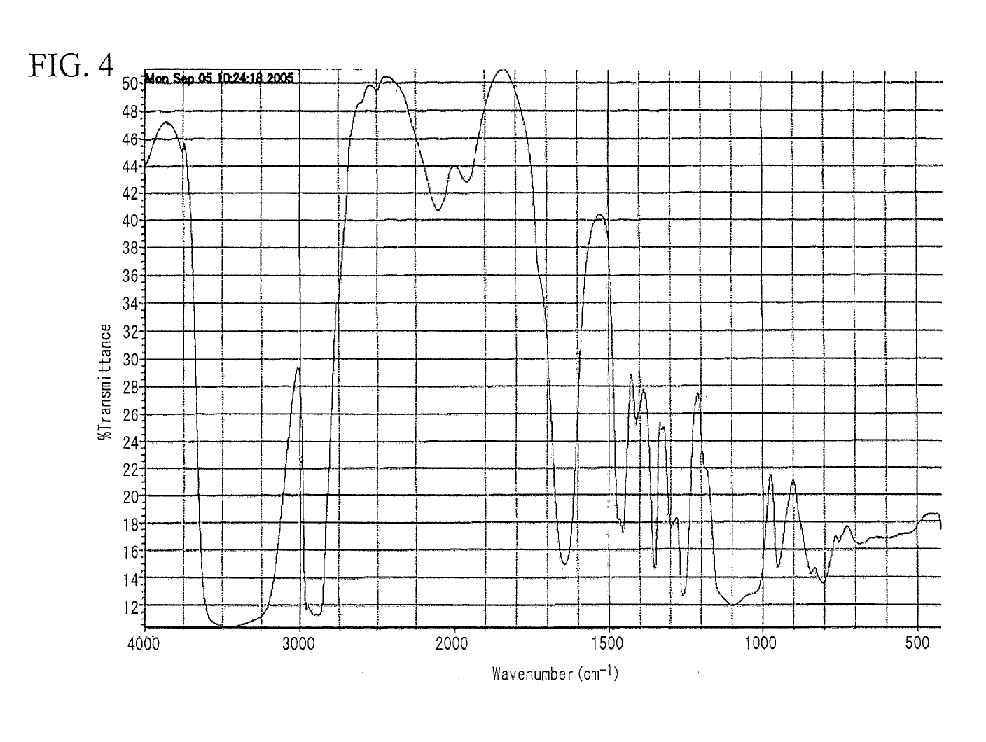

In addition, as a result of IR analysis after the deterioration test at 50° C., in both cases of the single material of the polyether-modified silicone of the aforementioned formula (II) and the mixture thereof with the buffer solution at pH 6, characteristic absorption at 1,720 cm⁻¹ was observed. In addition, as the pH decreased, the absorption strength increased. From the aforementioned observation, it can be seen that, in particular, under an acidic condition, the polyether-modified silicone of the aforementioned formula (II) was oxidation-decomposed, and a carbonyl-functional compound was easily produced. On the other hand, in both the case of the single material of the aforementioned methyl polyglycerol-modified silicone and the case of the mixture thereof with the buffer solution at pH 6, the characteristic absorption at 1,720 cm⁻¹ was hardly observed, and it can be seen that a carbonyl-functional compound was hardly produced (see FIG. 1 to FIG. 4).

Evaluation of Viscosity

The degree of viscosity of the silicone-based surfactants used in the present invention was evaluated, as compared with the degree of viscosity of the silicone-based surfactants which are the same as the silicone-based surfactants of the present invention with the exception that the OH as the terminal group of the aforementioned silicone-based surfactant is not alkylated.

Glycidol was subjected to a ring-opening polymerization in the presence of glycerol monoallyl alcohol (molar ratio of glycerol monoallyl alcohol:glycidol=1:12), without using glycidyl methyl ether or glycidyl ethyl ether, and thereby, an allyloxyethoxy-terminal polyglycerol was obtained. The obtained polyglycerol exhibited fluidity when it was heated, but hardly exhibited fluidity at room temperature. In addition, the obtained polyglycerol had increased viscosity, as compared with the viscosity of the allyloxyethoxy-terminal methyl polyglycerol and the allyloxyethoxy-terminal ethyl polyglycerol, and was extremely difficultly taken out from the reactor.

Subsequently, the aforementioned polyglycerol was subjected to an addition reaction with a copolymer of polydimethylsiloxane and polymethylhydrogensiloxane represented by the aforementioned formula (I) to obtain a polyglycerol-modified silicone. The obtained silicone exhibited fluidity when it was heated, but hardly exhibited fluidity at room temperature. The obtained silicone had increased viscosity, as compared with a methyl polyglycerol-modified silicone and an ethyl polyglycerol-modified silicone which were synthesized by independently addition-reacting the allyloxyethoxy-terminal methyl polyglycerol and the allyloxyethoxy-terminal ethyl polyglycerol synthesized in reference examples with the polydimethylsiloxane-polymethylhydrogensiloxane copolymer represented by the aforementioned formula (I) in the same manner as described above. The obtained silicone was extremely difficult to be taken out from the reactor.

### INDUSTRIAL APPLICABILITY

The emulsions of the present invention and the emulsions produced by the present invention are useful as water repellent agents, mold releasing agents, lubricants, fiber treatment agents, leather treatment agents, artificial leather treatment agents, cosmetic additives, cosmetics, glazing agents, defoaming agents, surface treatment agents, coating agents, or the like. In particular, the emulsions are suitable as raw materials for cosmetics, and preferably used as additives for use in cosmetics or cosmetics as they are.

1. An emulsion comprising:
(A) an organopolysiloxane;
(B) a silicone-based surfactant represented by general formula (1):
R²₃SiO(R¹₂SiO)ₘ(R¹YSiO)ₙSiR²₃ (1)
wherein
each R¹ independently represents a hydrogen atom or a substituted or non-substituted monovalent hydrocarbon group;
each Y independently represents a group represented by the following general formula (2):
—CₐH₂ₐ(OC₂H₄)_{b}(OC₃H₆)_{c}—O—(B)_{d}-A (2)
wherein
A represents a terminal group represented by the following formula (3), (4) or (5): 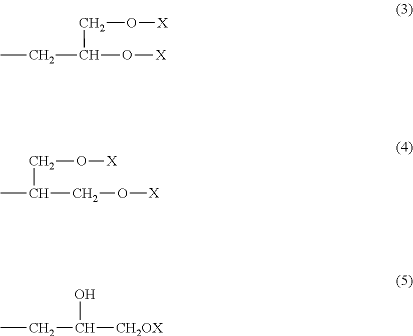
in each of the formulae, X represents a hydrogen atom or independently represents a substituted or non-substituted monovalent hydrocarbon group containing no aliphatic unsaturated bond, with not more than 20 carbon atoms; and at least one of the Xs is the hydrocarbon group;
B represents a moiety represented by the following formula (6), (7), (8) or (9): 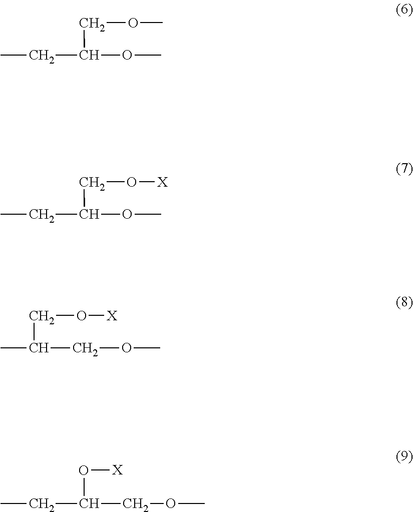
wherein
X is the same as described above;
(OC₂H₄) and (OC₃H₆) are arranged in any one of a random type, a block type, and an alternative type, or a mixed type thereof;
a ranges from 2 to 15;
b ranges from 0 to 100;
c ranges from 0 to 100;
d ranges from 0 to 500,
m ranges from 0 to 50;
n ranges from 0 to 20; and
R² represents R¹ or X, with the proviso that when n is 0, at least one R² represents X; and
(C) an aqueous medium.

2. The emulsion according to claim 1, wherein in general formula (1), m ranges from 0 to 6 and n ranges from 0 to 3.

3. The emulsion according to claim 1, wherein in general formula (1), at least 15% of the Xs of the terminal groups is the hydrocarbon group.

4. The emulsion according to claim 1, wherein the organopolysiloxane (A) has a viscosity ranging from 50 to 3,000 mPa·s at 25° C.

5. A method for producing the emulsion as recited in claim 1, characterized by comprising emulsifying a mixture of the organopolysiloxane (A) and the silicone-based surfactant (B), which is obtained by synthesizing the silicone-based surfactant (B) represented by general formula (1) in the organopolysiloxane (A).

6. The method for producing the emulsion according to claim 5, wherein the silicone-based surfactant (B) is synthesized by subjecting a silicon atom-bonding hydrogen atom-containing siloxane and a terminal double bond-containing compound to a hydrosilylation reaction in the presence of a catalyst for use in a hydrosilylation reaction.

7. The method for producing the emulsion according to claim 5, wherein the silicon atom-bonding hydrogen atom-containing siloxane is represented by general formula (1′):
R²₃SiO(R¹₂SiO)ₘ(R¹HSiO)ₙSiR²₃ (1′)
wherein
each R¹ independently represents a hydrogen atom or a substituted or non-substituted monovalent hydrocarbon group;
m ranges from 0 to 50;
n ranges from 0 to 20; and
R² represents R¹ or H, with the proviso that when n is 0, at least one R² represents H.

8. The method for producing the emulsion according to claim 7, wherein m ranges from 0 to 6 and n ranges from 0 to 3.

9. The method for producing the emulsion according to claim 6, wherein the terminal double bond-containing compound is represented by the following general formula (2′):
CH₂═CH—C_{a′}H_{2a′}(OC₂H₄)_{b}(OC₃H₆)_{c}—O—(B)_{d}-A (2′)
wherein
A represents a terminal group represented by the following formula (3), (4) or (5): 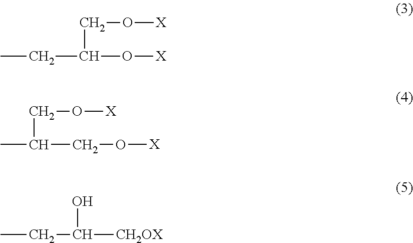
in each of the formulae, X represents a hydrogen atom or independently represents a substituted or non-substituted monovalent hydrocarbon group containing no aliphatic unsaturated bond, with not more than 20 carbon atoms; and at least one of the Xs is the aforementioned hydrocarbon group;
B represents a moiety represented by the following formula (6), (7), (8) or (9): 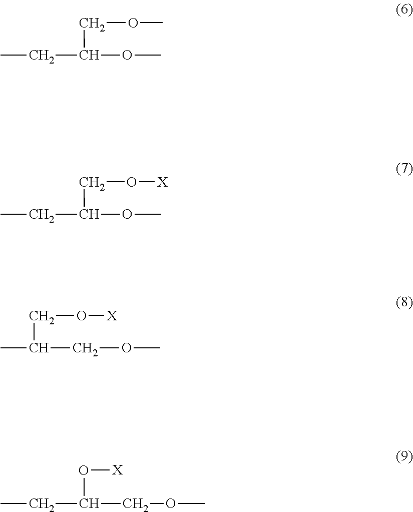
wherein
X is the same as described above;
(OC₂H₄) and (OC₃H₆) are arranged in any one of a random type, a block type, and an alternative type, or a mixed type thereof;
a′ ranges from 0 to 13;
b ranges from 0 to 100;
c ranges from 0 to 100; and
d ranges from 0 to 500.

10. The method for producing the emulsion according to claim 9, wherein at least 15% of the Xs of the terminal groups is the hydrocarbon group.

11. The method for producing the emulsion according to claim 5, wherein the organopolysiloxane (A) has a viscosity ranging from 50 to 3,000 mPa·s at 25° C.

12. An emulsion obtainable by the method as recited claim 5.

13. A cosmetic raw material comprising emulsion as recited in claim 1.

14. The emulsion according to claim 2, wherein in general formula (1), at least 15% of the Xs of the terminal groups is the hydrocarbon group.
